(19) United States
(12) Patent Application Publication
(10) Pub. No.: US 2022/0001050 A1
(43) Pub. Date: Jan. 6, 2022

(54) DISINFECTION TOWER

(71) Applicant: Dolphin Care ApS, Hellerup (DK)

(72) Inventor: Jørn TERKELSEN, Hørsholm (DK)

(73) Assignee: Dolphin Care ApS, Hellerup (DK)

(21) Appl. No.: 17/283,342

(22) PCT Filed: Mar. 22, 2019
(86) PCT No.: PCT/EP2019/057219

§ 371 Date: Apr. 7, 2021

(30) Foreign Application Priority Data
Oct. 29, 2018 (EP) 18203144.3

(51) Int. Cl. A61L 2/10

(52) U.S. Cl. CPC A61L 2/10; A61L 2202/11; A61L 2202/25; A61L 2202/14; A61L 2202/16

(57) ABSTRACT

A disinfection tower adapted to receive current when in operation including (a) a support house having a space inside the house adapted to include an exchangeable current supply unit, wherein the house has an elongated shape having a circumference, a top part and a bottom part opposite each other, (b) multiple elongated UVC light sources adapted for radiating microorganisms, wherein each UCV light source is fixed and disposed outside the house at a desired distance from the circumference of the house and in a longitudinal direction relative to the house, and wherein each UVC light source is disposed with a suitable distance configured to eliminate overheating of each UVC light source.

### FIELD OF THE INVENTION

The present invention relates to a disinfection tower, as well as a method of removing microorganisms by using the disinfection tower. Furthermore, the present invention relates to use of the disinfection tower for removing microorganisms, such as bacteria.

### BACKGROUND OF THE INVENTION

In 1877 British scientists Downes and Blunt discover the ability of sunlight to prevent microbial growth. It is later show that the ability of light to inactivate microorganisms is dependent on the dose (intensity×time) and wavelength of radiation and the sensitivity of the specific type of microorganism.

Since then UVC light is known to reduce or kill bacteria in air, liquids, e.g. water, and on surfaces.

Radiation with UVC light kill or inactivate microorganisms like bacteria, viruses, molds, and other pathogens by destroying nucleic acids and disrupting their DNA/RNA.

UVC light can replace todays use of harmful strong detergents and chemicals, that are used for disinfecting patient wards, surgery rooms, toilets, etc. Using UVC light will take between 15 minutes and 2 hours compared to todays 12 to 18 hours using strong detergents and chemicals.

### SUMMARY OF THE INVENTION

The present inventors have realized that there is a need for more efficient disinfection of rooms, in particular patient bed rooms and rooms for surgery in a hospital or similar facilities. There are no effective devices on the market that can effectively reduce and even completely remove microorganisms from closed rooms, in particular hospital rooms. UVC light is complex and the light cannot move around corners, which means that the UVC light must hit the microorganism at all times. In addition, the major number of microorganisms are present from the floor level to about 100 cm and consequently a high dose of UVC light is needed in that area and at the same time the microorganism should not be able to hide behind walls or around corners.

The new and inventive construction provides an effective reduction and complete removal of microorganisms without the need for toxic chemicals and risk for humans working with such chemicals. Moreover, the disinfection tower of the present invention makes it possible to clean such rooms much faster within a few hours, or sometimes even within less than one hour, making it much more effective and consequently resources can be saved.

A further challenge has been the current supply, since a lot of wires are needed to supply current to the UVC lamps, when at the same time the high of the tower is important to reach a sufficient number of microorganisms. The present construction makes it possible to hide all wires and the like for supplying current to the UVC lamps inside the disinfection tower, and at the same time provide a stable tower that can be moved by people and staff working at the hospitals.

The present invention concerns a disinfection tower adapted to receive current when in operation comprising

(a) a support house having a space inside the house adapted to comprise an exchangeable current supply unit, wherein the house has an elongated shape having a circumference and a top part and a bottom part opposite each other,

(b) multiple elongated UVC light sources adapted for radiating microorganisms, wherein each UCV light source is fixed and disposed outside the house at a desired distance from the circumference of the house and in a longitudinal direction relative to the house, and wherein each UVC light source is disposed with a suitable distance configured to eliminate overheating of each UVC light source.

In an embodiment the exchangeable current supply unit is disposed inside the support house. Such support house can have any shape as long as it is a tower, for instance the support house is cylindrical or is polygon shaped, such as decagon shaped. Furthermore, the support house is made of a UVC resistant material, such as steel, e.g. stainless steel.

In a further embodiment the disinfection tower of the present invention comprises a further UVC light source at the bottom part of the house, wherein the further UVC light source is adapted to radiate microorganisms at a floor under the bottom of the tower.

In a still further embodiment each UVC light source is adapted to provide UVC light at 250-260 nm, preferably at 254 nm. Preferably the UVC light source is a UVC lamp and the disinfection tower typically comprises from 8-20 UVC lamps, preferably 8-12 UVC lamps.

In a further embodiment the disinfection tower of the present invention comprises at least 4 wheels at the bottom part for stabilizing the tower and for easy transportation of the tower. Typically, the tower is a portable stand-alone device.

In a still further embodiment the disinfection tower of the present invention is suitable for functioning inside a closed room, such as a hospital room or a bed room, e.g. a room for surgery.

In a further embodiment a ventilation unit for cooling the current supply is located inside the house of the tower, preferably at the top part of the house.

In a still further embodiment the disinfection tower of the present invention comprises at least one satellite unit having a UVC light source and adapted to receive current from the current supply of the tower.

In a further embodiment the disinfection tower of the present invention comprises a control panel for operating the tower, such as a control panel disposed adjacent the top part of the house.

In a further aspect the present invention concerns a method of removing microorganisms, such as multi resistant bacteria, from a closed room, preferably a room for surgery, patient rooms and treatment rooms, comprising placing the disinfection tower of the present invention in the room to be disinfected and supplying current to the disinfection tower.

In an embodiment the current is turned on for at least 5 minutes, such as from 15 to 240 minutes.

In a still further aspect the present invention concerns use of the disinfection tower of the present invention in a room for removing microorganisms from the room.

The present invention provides these advantages with the described solution.

Further objects and advantages of the present invention will appear from the following description, and claims.

### DESCRIPTION OF THE INVENTION

In a broad aspect the present invention concerns a disinfection tower adapted to receive current when in operation comprising

(a) a support house having a space inside the house adapted to comprise an exchangeable current supply unit, wherein the house has an elongated shape having a circumference, and a top part and a bottom part opposite each other,

(b) multiple elongated UVC light sources adapted for radiating microorganisms, wherein each UCV light source is fixed and disposed outside the house at a desired distance from the circumference of the house and in a longitudinal direction relative to the house, and wherein each UVC light source is disposed with a suitable distance configured to eliminate overheating of each UVC light source.

The disinfection tower is preferably constructed of materials that can resist UVC light with a wave length of 250-260 nm, such as metals, e.g. steel. The disinfection tower can in principle have any suitable height, width and depth as long as it can be moved by a person and fit into the room for disinfection. The tower has an elongated shape and thus is higher than the width and the depth, and is adapted so it can stand upright. Typically, the tower is elongated and have a polygon shaped or cylindrical cross section all though the cross section may also be square or rectangular. In particular the tower comprises the support house which supports the tower and is constructed to contain an exchangeable current supply unit.

In a further embodiment the exchangeable current supply unit is disposed inside the support house. Such support house can have any shape as long as it is a tower, for instance the support house is cylindrical, or decagon shaped. Furthermore, the support house is made of a UVC resistant material, such as steel, e.g. stainless steel.

Usually one elongated UVC light source, such as a UVC lamp, cannot provide sufficient UVC light to disinfect a room, and thus multiple elongated UVC light sources are used. The elongated UVC light sources must be spaced apart with a sufficient distance to avoid overheating of the UVC light sources and at the same time there should be sufficient UVC light sources to provide efficient reduction of microorganism and disinfection of the particular room to be disinfected. Typically, the UVC light source is a UVC lamp and the disinfection tower typically comprises from 8-20 UVC lamps, preferably 8-16 UVC lamps, such as 8-12 UVC light sources, e.g. 8-12 UVC lamps. Preferably, each UVC light source is adapted to provide UVC light at 250-260 nm, and the optimal disinfection is obtained at a wave length of 254 nm.

The dimensions of the tower and also the UVC lamps can be varied depending on the room to be treated and disinfected, however, the length of the elongated UVC lamps are preferably at least 100 cm and the tower is constructed so that it can support the UVC lamps. For a typical hospital room, the elongated UVC lamps are from 100-200 cm, and typically from 100-150 cm, such as from 120-140 cm. The present invention has been tested with different length of the UVC lamps and at least 100 cm is necessary to kill all microorganisms within sufficient time, such as 15 min to about 4 hours.

The disinfection tower of the present invention can be placed in a closed room and the UVC lamps turned on for sufficient time to kill all bacteria and can then be moved to disinfect the area under the tower which has not been treated with UVC light. However, in a further embodiment the disinfection tower of the present invention comprises a further UVC light source at the bottom part of the house, wherein the further UVC light source is adapted to radiate microorganisms at a floor area underneath the tower. Typically, one UVC lamp is provided at the bottom part of the tower.

The disinfection tower of the present invention is particularly useful for disinfection of a closed room. Typically, the dimensions of the room correlates with the dimensions of the tower and the UVC lamps to provide efficient reduction of microorganisms. The present invention is in particular suited for rooms with high number of different microorganisms, such as a hospital room or a bed room for patients in a hospital. Another preferred use is for disinfection of a room for surgery. As can be appreciated any closed room can be disinfected with the tower of the present invention, and this can be done in a reduced time compared to the known methods of cleaning rooms such as rooms in hospitals.

Due to the many wires of the current supply unit necessary to provide sufficient current to operate the tower of the present invention a ventilation unit for cooling the current supply unit when heated during operation is located inside the house of the tower. Such ventilation unit can be located any suitable place, such as at the top or bottom of the support house, preferably it is located at the top part of the house, which has proved to provide the most efficient cooling of the current supply unit during operation.

The disinfection tower of the present invention typically comprises at least 4 wheels at the bottom part for stabilizing the tower and for easy transportation of the tower. Typically, 4 or 5 wheels are sufficient to keep the tower stable.

Preferably, the tower is a portable stand-alone device, which means that it can be moved to the place for disinfection whenever it is needed and plugged in to receive current, and can be stored when not in use, and can be transported and operated by one person only.

Some rooms may have areas, such as room dividing or are not square or rectangular in shape, which means that in order to obtain complete and efficient reduction of microorganisms one or more satellite units having a UVC light source, typically one UVC lamp, and adapted to receive current from the current supply of the tower can be placed in the areas not covered by the main tower of disinfection. This combined disinfection tower and satellite provides a highly efficient device for removing microorganisms. The satellite typically has the shape of a tower and the same or similar height as the disinfection tower, although is smaller in circumference. The satellite typically comprises a metal grid supporting the satellite and providing protection for the UVC lamp.

In order for a person to operate the tower of the present invention and optionally the satellite(s) a control panel is provided for setting the time of UVC light and the start of disinfection so that the person operating the tower can get out of the room to be disinfected before the UVC light is switched on. The control panel can be disposed any suitable place on the tower, however is typically disposed adjacent the top part of the support house.

In a further aspect the present invention concerns a method of removing microorganisms from a closed room comprising placing the disinfection tower of the present invention in the room to be disinfected and supplying current to the disinfection tower. Any one of the above embodiments of the disinfection tower of the present invention, either individually or in combination, are embodiments of the present method.

In an embodiment the current is turned on for at least 5 minutes, such as from 15 to 240 minutes, such as from 30 to 180 minutes, such as 45 to 150 minutes, such as from 60 to 120 minutes. In a further embodiment when microorganisms are less sensitive to the UVC light the current is turned on for at least 120 minutes. The normal occurring antibiotic resistant microorganisms (Staphylococci, Pseudomonas, Enterobacteriae and enterococci) for which room disinfection is needed are very sensitive to UVC light for 5-15 minutes. Only few species will require a longer exposure time i.e mycobacteria and spores from Clostridium difficile.

In a still further embodiment the room is a room for surgery. Typically, a room for surgery in a hospital.

In a still further aspect the present invention concerns use of the disinfection tower of the present invention in a room for removing microorganisms from the room.

The term “portable and stand-alone” as used herein means a device which can be transported by the user, such as people working at a hospital, and plugged-in (for current supply) at any desired place, such as in a hospital room for sick people, a room for surgery, an office in a building.

The invention will now be described more fully with reference to the appended drawings illustrating typical embodiments of the air sterilizer unit of the present invention.

These drawings are by no means limiting the scope of the present invention and are only intended to guide the skilled person for better understanding of the present invention.

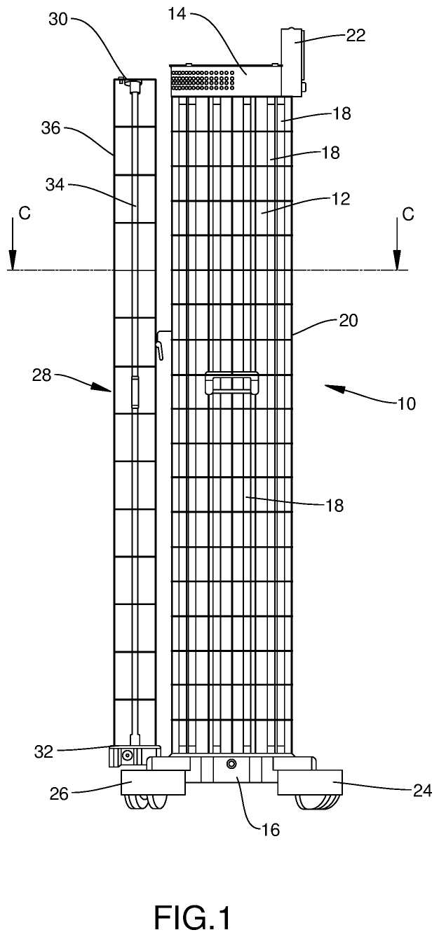

FIG. 1 illustrates a side view of a disinfection tower (10) adapted to receive current when in operation. The disinfection tower (10) as shown is cylindrical (or substantially cylindrical) having a support house (12) which support house (12) has a space inside (not shown) the house adapted to comprise an exchangeable current supply unit. The tower (10) and house (12) have an elongated shape having a circumference (not shown), a top part (14) and a bottom part (16) which top, and bottom are opposite each other. Moreover, multiple elongated UVC lamps (18) are fixed and disposed outside the house (12) at a desired distance from the circumference of the house (12) and in a longitudinal direction relative to the house (12). Furthermore, each UVC lamp (18) is disposed with a suitable distance configured to eliminate overheating of each UVC lamp. A metal grid (20) is arranged outside the tower and house to provide support and in particular to protect the UVC lamps (18). In this embodiment a control panel (22) is arranged at the top (14) to start and stop the disinfection tower (10) and set timing and dose of UVC light. In the bottom, wheels (24, 26), typically 4 wheels, are arranged to easily move the tower from storage to the room for disinfection. Here is also shown a satellite unit (28) having an elongated shape with a top part (30) and a bottom part (32) and an UVC lamp (34) protected by a metal grid (36). The satellite (28) is equipped with a platform at the bottom part (32) to be able to stand separate from the disinfection tower. The satellite (28) is separate from the tower when in use and can be stored together with the tower when not in use.

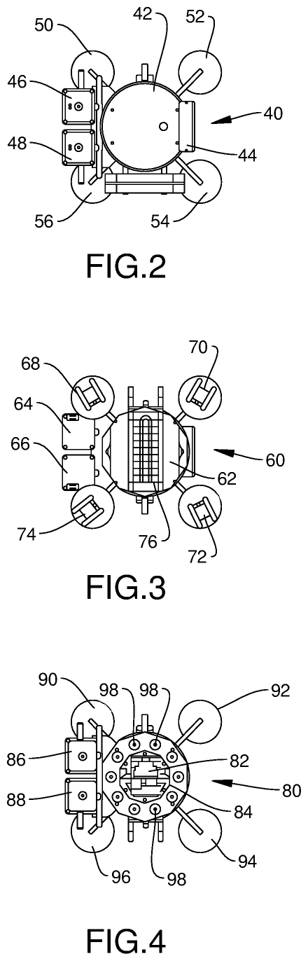

FIG. 2 illustrates the top view (40) of the disinfection tower of FIG. 1 showing the house (42) (which is decagon shaped), the control panel (44), two satellites (46, 48) and four wheels (50, 52, 54, 56).

FIG. 3 illustrates the bottom view (60) of the disinfection tower of FIG. 1 showing the decagon shaped house (62), two satellites (64, 66) and four wheels (68, 70, 72, 74). Furthermore, one UVC lamp (76) is located at the bottom and adapted to provide UVC light to the floor to be disinfected.

FIG. 4 illustrates a cross section of the tower of FIG. 1 along the line C-C. The disinfection tower (80) has a current supply unit (82) disposed in the center of the tower (80) and a decagon shaped mantle (84) comprises and protects the current unit (82). Further illustrated are the two satellites (86, 88), the four wheels (90, 92, 94, 96) and the UVC lamps (98).

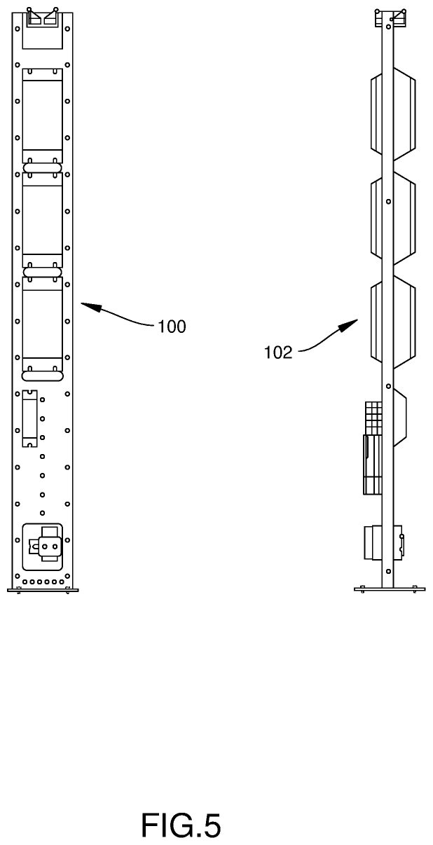

FIG. 5 illustrates the current supply unit seen from the front side (100) and as side-view (102) which current supply unit (100, 102) is adapted to fit inside the disinfection tower house.

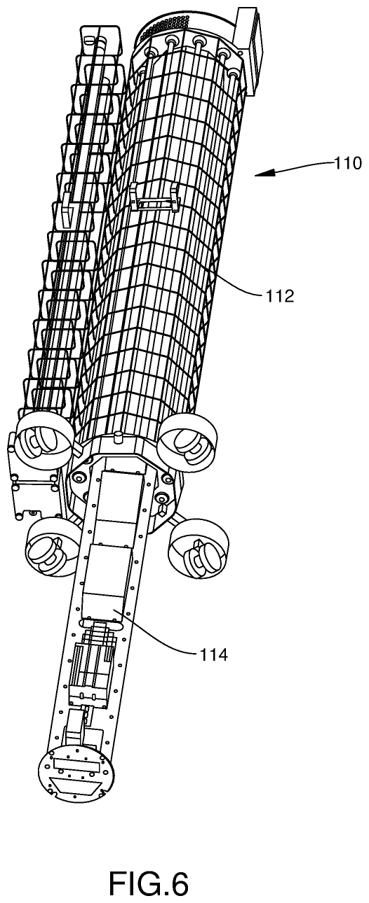

FIG. 6 illustrates the disinfection tower (110) of FIG. 1 seen from a perspective view from the bottom where the current supply unit (114) is inserted into the house (112). Once inserted into the house (112), the current supply unit (114) is sealed off and fixed by fastening means, such as screws to the house (112).

All references, including publications, patent applications and patents, cited herein are hereby incorporated by reference to the same extent as if each reference was individually and specifically indicated to be incorporated by reference and was set forth in its entirety herein.

All headings and sub-headings are used herein for convenience only and should not be construed as limiting the invention in any way.

Any combination of the above-described elements in all possible variations thereof is encompassed by the invention unless otherwise indicated herein or otherwise clearly contradicted by context.

Recitation of ranges of values herein are merely intended to serve as a short method of referring individually to each separate value falling within the range, unless other-wise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about”, where appropriate).

All methods described herein can be performed in any suitable order unless otherwise indicated herein or otherwise clearly contradicted by context.

The terms “a” and “an” and “the” and similar referents as used in the context of de-scribing the invention are to be construed to insert both the singular and the plural, unless otherwise indicated herein or clearly contradicted by context. Thus, “a” and “an” and “the” may mean at least one, or one or more.

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

Throughout the description when “selected from” or “selected from the group consisting of” is used it also means all possible combinations of the stated terms, as well as each individual term.

The citation and incorporation of patent documents herein is done for convenience only and does not reflect any view of the validity, patentability and/or enforceability of such patent documents.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The features disclosed in the foregoing description may, both separately and in any combination thereof, be material for realizing the invention in diverse forms thereof.

### EXPERIMENTALS

The disinfection tower as illustrated in FIGS. 1-6 was constructed with 10 UVC lamps each providing 254 nm UVC light, wherein each UVC lamp is disposed 55 mm from each other and the 10 UVC lamps are arranged at the circumference of the tower house. The tower house is about 175 cm in total height depending on the size of the wheels and top part closing and sealing of the tower house. Each UVC lamp has a length of about 156 cm and a diameter of about 15 mm Each UVC lamp has a Lamp Wattage of 145 W, a Lamp Current of 800 mA and a Lamp Voltage at High Frequency 182 V. The Physical Data are UV Output 253.7 nm (100 hr) 54 W, Intensity @ 1 m 410 μW/cm2 and a Rated Average Life*16000 hrs.

The decagon shaped house has a diameter of about 30 cm, which housing contains the current supply unit providing current of 0.68 A to each UVC lamp at its maximum when in use at Copenhagen University Hospital, Rigshospitalet in Denmark.

The 10 UVC lamps are elongated and the bottom part closest to the floor of the room being disinfected is about 11 cm above the floor. The bottom of the tower is further equipped with one UVC lamp providing 254 nm UVC light which lamp is located about 7 cm above the floor.

This disinfection tower has been tested by the Department of Clinical Microbiology at Copenhagen University Hospital, Rigshospitalet in Denmark in several rooms.

Various rooms at two hospitals in Denmark have been tested with the disinfection tower as illustrated in FIGS. 1-6. The rooms vary form 8 m² to 100 m², where the 8 m² typically are toilets and 100 m² rooms are bed rooms for patients. In the toilets the disinfection tower typically runs for 5 minutes to kill all microorganisms. In bed rooms up to 100 m² suited for many patients the disinfection tower runs for 15 min for some bacteria and microorganisms and for 2 hours if special multi resistant microorganisms are present. In general, the disinfection tower of FIGS. 1-6 has a reach of 5 meters, which means that in some instances it may be necessary to make to runs to cover a larger room with more than 5 meters from the tower to the walls of the room.

Typical bed rooms are from 15 to 50 m2 and the disinfection tower as illustrated in FIGS. 1-6 can handle rooms up to 50 m2 in one run. The typical rooms of 50 m2 are not more than 2.5 meters to the top (ceiling) and no more than 5 meters form any corner to the tower when correctly placed in the center of the room.

The test results are as follows:

Two scientific studies done by the Department of Clinical Microbiology, Copenhagen University Hospital, Rigshospitalet in Denmark (in confidentiality) showed a log 5-6 reduction of resistant facultative and aerobic bacteria at a 5-meter (16.4-foot) distance from the unit (disinfection tower, such as illustrated in FIGS. 1-6) after 15 minutes of irradiation.

In the first study, testing focused on swabs taken from surfaces in four different outpatient clinics for patients suffering from Cystic Fibrosis. Swabs were taken before and after 30 minutes in-room exposure to high UV-C radiation. The bacteria present were virtually eliminated.

In the second study, in vitro samples were placed within 5 meters of a source of UV-C radiation. 99.999% of the bacteria present were killed after 15 minutes of exposure to radiation (bacteria tested included Enterococcus faecium (VRE), Enterococcus faecalis, Staphylococcus aureus, Acinetobacter baumannii, Klebsiella pneumoniae, and Stenotrophomonas maltophilia). This level of elimination is more than sufficient for the purpose of room disinfection.

Results

Vegetative bacteria are much more sensitive to UVC light than spores as expected. The longer exposure time to UVC the greater is the effect on both vegetative bacteria and spores and the closer the bacteria are to the UVC source the greater is the effect of UVC light.

Effect of UVC on vegetative bacteria after 15 minutes UVC exposure is shown in Table 1. The number is given as the average value of the two experiments. The highest start concentration of about 108 Colony-forming unit/cm² (CFU/cm²) on the plates are shown in the first column. The next columns show the number of bacteria remaining on the plates at 3 meters, 4 meters and 5 meters from the UVC source after 15 minutes exposure to UVC.

Gram negative bacteria are reduced about 8 log 10 at 3 m, 6 log 10 at 4 m and 6 log 10 at 5 m. A ten-fold reduction in the start concentration of bacteria resulted in a 7 log 10 reduction at 5 m.

Gram positive bacteria were less susceptible to UVC light than Gram negative bacteria. The reduction was around 6 log 10 at 3 m and around 5 log 10 at 5 m. A ten-fold reduction of the start concentration of bacteria resulted in 6 log 10 reduction at 3 m and 5 log 10 at 5 m. Vancomycin resistant Enterococcus faecium seem to be less sensitive to UVC than other Gram positive bacteria.

TABLE 1

| | Highest concentration of | | | |
|---|---|---|---|---|
| | bacteria on the plates | | Concentration at 3 meters | |
| | before exposure to UVC | | after exposure to UVC | |
| Bacteria | (average value) | | (average value) | |
| Staphylococcus aureus | 6.9 × 10⁸ | CFU/cm² | 1.2 × 10² | CFU/cm² |
| Enterococcus faecalis | 2.4 × 10⁷ | CFU/cm² | 1 × 10² | CFU/cm² |
| Enterococcus faecium (VRE) | 3.2 × 10⁸ | CFU/cm² | 5 × 10² | CFU/cm² |
| Klebsiella pneumoniae | 1.4 × 10⁸ | CFU/cm² | 0 | CFU/cm² |
| Acinetobacter baumanii | 1.9 × 10⁸ | CFU/cm² | 0 | CFU/cm² |
| Stenotrophomonas maltophilia | 3.6 × 10⁷ | CFU/cm² | 0 | CFU/cm² |
| | Concentration at 4 meters | | Concentration at 5 meters | |
| | after exposure to UVC | | after exposure to UVC | |
| Bacteria | (average value) | | (average value) | |
| Staphylococcus aureus | 3 × 10² | CFU/cm² | 9.8 × 10² | CFU/cm² |
| Enterococcus faecalis | 1.3 × 10³ | CFU/cm² | 2.4 × 10³ | CFU/cm² |
| Enterococcus faecium (VRE) | 3.8 × 10³ | CFU/cm² | 4 × 10³ | CFU/cm² |
| Klebsiella pneumoniae | 1 × 10² | CFU/cm² | 7.5 × 10¹ | CFU/cm² |
| Acinetobacter baumanii | 5 × 10¹ | CFU/cm² | 5 × 10¹ | CFU/cm² |
| Stenotrophomonas maltophilia | 7.5 × 10¹ | CFU/cm² | 7.5 × 10¹ | CFU/cm² |

Effect of UVC on spores of Bacillus cereus after 90 minutes exposure was a reduction of 6 log₁₀ at 2 meter, 3 meter and 4 meter from the UVC source (Table 2). The reduction of C. difficile spore after 90 min UVC exposure at 2, 3 or 4 meters were about 1 login (Table 2).

TABLE 2

| | Highest concentration of | |
|---|---|---|
| | bacteria on the plates | Concentration at 2 meters |
| | before exposure to UVC | after exposure to UVC |
| Bacteria | (average value) | (average value) |
| Bacillus cereus | 5.5 × 10⁷ CFU/cm² | 1.1 × 10¹ CFU/cm² |
| Clostridium difficile | 3.5 × 10⁴ CFU/cm² | 2.2 × 10³ CFU/cm² |
| | Concentration at 3 meters | Concentration at 4 meters |
| | after exposure to UVC | after exposure to UVC |
| Bacteria | (average value) | (average value) |
| Bacillus cereus | 1.5 × 10¹ CFU/cm² | 4 × 10¹ CFU/cm² |
| Clostridium difficile | 3.1 × 10³ CFU/cm² | 1.7 × 10³ CFU/cm² |

The field test was done in three rooms in an outpatient clinic for patients with cystic fibrosis. The UVC exposure time in-between imprints before and after exposure was 30 min. Imprints were taken from 5 standardized spots in each room. The Results are shown in table 3. Sample were taken 8 times from room 1, 5 from room 2 and 4 times from room 3. All numbers are given as CFU/imprint plate (20 cm²).

The results for each room are shown in FIG. 3 a-c. Even though the contamination at the spots where same are taken before UVC a clear reduction in the number of bacteria was found. Chairs were the most contaminated spots and also here a clear reduction of bacteria were seen. (n is number of samples).

TABLE 3

| | Room 1 (n = 8) | | Room 2 (n = 5) | | Room 3 (n = 4) | |
|---|---|---|---|---|---|---|
| | Before | | Before | | Before | |
| Spot | UVC | After UVC | UVC | After UVC | UVC | After UVC |
| Chair 1 | 12, 24, | 0, 0, | 0, 8, | 0, 0, | 18, 53, | 0, 0, |
| | 36, 8, | 0, 0, | 21, 5, | 9, 0, | 288, 276 | 1, 2 |
| | 2, 356, | 0, 6, | 62 | 0 | | |
| | 3, 54 | 3, 0 | | | | |
| Chair 2 | 269, 57, | 3, 0, | 4, 9, | 3, 0, | 14, 348, | 0, 0, |
| | 2, 3, | 0, 2, | 157, 2, | 4, 0, | 21, 47 | 1, 2 |
| | 0, 119, | 1, 0, | 3 | 3 | | |
| | 7, 34 | 2, 2 | | | | |
| Patients | 9, 26, | 3, 0, | 15, 0, | 0, 1, | 11, 10, | 1, 0, |
| table | 3, 2, | 0, 0, | 4, 2, | 10, 10, | 81, 50 | 0, 5 |
| | 5, 198, | 0, 0, | 3 | 2 | | |
| | 6, 68 | 2, 0 | | | | |
| Work table | 5, 30, | 0, 0, | 3, 25, | 5, 0, | 8, 6, | 0, 0, |
| | 17, 2, | 0, 1, | 24, 1, | 0, 0, | 26, 16 | 0, 0 |
| | 1, 70, | 0, 0, | 21 | 1 | | |
| | 6, 11 | 0, 0 | | | | |
| Door | 0, 3, | 1, 2, | 2, 1, | 0, 0, | 10, 2, | 8, 0, |
| handle | 12, 1, | 0, 0, | 151, 0, | 11, 0, | 54, 16 | 0, 0 |
| | 3, 52, | 3, 0, | 9 | 1 | | |
| | 19, 6 | 1, 0 | | | | |
| Avarage | 39 | 1 | 22 | 2 | 63 | 1 |

1-16. (canceled)

17. A disinfection tower adapted to receive current when in operation comprising:
(a) a support house having a space inside the house adapted to comprise an exchangeable current supply unit, wherein the house has an elongated shape having a circumference and a top part and a bottom part opposite each other, and
(b) multiple elongated UVC light sources adapted for radiating microorganisms, wherein each UCV light source is fixed and disposed outside the house at a desired distance from the circumference of the house and in a longitudinal direction relative to the house, and wherein each UVC light source is disposed with a suitable distance configured to eliminate overheating of each UVC light source.

18. The disinfection tower of claim 17, wherein the exchangeable current supply unit is disposed inside the house.

19. The disinfection tower of claim 17, wherein the support house is polygon shaped, such as decagon shaped.

20. The disinfection tower of claim 17, comprising a further UVC light source at the bottom part of the house, wherein the further UVC light source is adapted to radiate microorganisms at a floor under the bottom of the tower.

21. The disinfection tower of claim 17, wherein each UVC light source is adapted to provide UVC light at 250-260 nm.

22. The disinfection tower of claim 17, wherein the support house is made of a UVC resistant material.

23. The disinfection tower of claim 17 comprising from 8-20 UVC lamps.

24. The disinfection tower of claim 17 comprising at least 4 wheels at the bottom part for stabilizing the tower and for easy transportation of the tower.

25. The disinfection tower of claim 17, which is a portable stand-alone device.

26. The disinfection tower of claim 17, which is suitable for functioning inside a closed room.

27. The disinfection tower of claim 17, wherein a ventilation unit for cooling the current supply is located inside the house of the tower.

28. The disinfection tower of claim 17, further comprising at least one satellite unit having a UVC light source and adapted to receive current from the current supply of the tower.

29. The disinfection tower of claim 17, further comprising a control panel for operating the tower.

30. A method of removing microorganisms, from a closed room, comprising: placing the disinfection tower of claim 17 in the room to be disinfected, and supplying current to the disinfection tower.

31. The method of claim 30, wherein the current is turned on for at least 5 minutes.
